(19) United States
(12) Patent Application Publication
(10) Pub. No.: US 2015/0044138 A1
(43) Pub. Date: Feb. 12, 2015

(54) METHOD FOR IMPROVING FUNCTIONAL SYNAPTIC CONNECTIVITY

(71) Applicant: N.V. Nutricia, Zoetermeer, (NL)

(72) Inventors: Marieke Lansbergen, Utrecht (NL); Martine Groenendijk, Utrecht (NL); Patrick Joseph Gerardus Hendrikus Kamphuis, Utrecht (NL); Mattheus Cornelis De Wilde, Utrecht (NL)

(73) Assignee: N.V. Nutricia, Zoetermeer (NL)

(21) Appl. No.: 14/381,918

(22) PCT Filed: Mar. 4, 2013
(86) PCT No.: PCT/NL2013/050135

§ 371 Date: Aug. 28, 2014

(30) Foreign Application Priority Data
Mar. 2, 2012 (NL) PCT/NL2012/050129
Jul. 6, 2012 (NL) PCT/NL2012/050487

(51) Int. Cl. A61K 31/7072; A61K 31/14; A61K 31/675; A61K 31/714; A61K 49/22; A61K 31/519; A61K 33/04; A61K 31/375; A61K 31/355; A61K 31/202; A61K 31/685

(52) U.S. Cl. CPC A61K 31/7072; A61K 31/202; A61K 31/14; A61K 31/675; A61K 31/714; A61K 31/685; A61K 31/519; A61K 33/04; A61K 31/375; A61K 31/355; A61K 49/22
USPC 424/9.2; 514/51; 424/702

(57) ABSTRACT

The invention relates to a composition comprising: (i) one or more of uridine and cytidine, or salts, phosphates, acyl derivatives or esters thereof; and (ii) a lipid fraction comprising at least one of docosahexaenoic acid (22:6; DHA), eicosapentaenoic acid (20:5; EPA) and docosapentaenoic acid (22:5; DPA), or esters thereof, for use in preserving or improving functional synaptic connectivity and/or preserving brain network organization in a subject in need thereof.

### FIELD OF THE INVENTION

The invention is in the field of medical nutrition and more particularly relates to a composition for use in improving or preserving functional synaptic connectivity and/or improving or preserving brain network organization, in particular for use in improving functional synaptic connectivity impairment in the brain areas and/or improving or preserving brain network organization of a subject, in particular a subject suffering from compromised functional connectivity and/or suffering from compromised brain network organization.

More particularly, the invention relates to a composition for use in improving or preserving functional synaptic connectivity and/or brain network organization, in particular for use in improving functional synaptic connectivity impairment in the brain areas and/or improving or preserving brain network organization of a subject, in particular a subject suffering from or at risk of a neurological disorder, in particular neurodegenerative disorder and/or compromised functional connectivity and/or compromised brain network organization, particularly patients suffering from or at risk of Alzheimer's disease.

### BACKGROUND DESCRIPTION

Brain connectivity is paramount for investigating how neurons and neural networks process information, and it is at the basis of many neurodegenerative implications. In the field, there is a fundamental distinction between the pattern of anatomical links (‘anatomical connectivity’), statistical dependencies of neurophysiolocial or other time series coming from different brain regions (‘functional connectivity’) and causal interactions (‘effective connectivity’), all within the general concept of brain connectivity [Horwitz (2003)].

Anatomical connectivity refers to a network of physical or structural (synaptic) connections linking sets of neurons or neuronal elements, as well as their associated structural biophysical attributes encapsulated in parameters such as synaptic strength or effectiveness. The physical pattern of anatomical connections is relatively stable at shorter time scales (seconds to minutes). At longer time scales (hours to days), structural connectivity patterns are likely to be subject to significant morphological change and plasticity.

Functional connectivity reflects the functional interactions between the underlying brain regions. It is defined as the “temporal correlations between spatially remote neurophysiological events” (Lee et al., 2003 and Friston et al., 1993a). In general, a method for assessing functional connectivity captures deviations from statistical independence between distributed and often spatially remote neuronal units. Statistical dependence may be estimated by measuring correlation or covariance, spectral coherence or phase-locking. Functional connectivity is often measured between all elements of a system, regardless of whether these elements are connected by direct structural links. Unlike structural connectivity, functional connectivity is highly time-dependent. Statistical patterns between neuronal elements fluctuate on multiple time scales, some as short as tens or hundreds of milliseconds. It should be noted that functional connectivity does not make any explicit reference to specific directional effects or to an underlying structural model.

Effective connectivity may be viewed as the union of structural and functional connectivity, as it describes networks of directional effects of one neural element over another.

Like functional connectivity, the concept of brain network organization is known in the field, and addressed in the art: D. S. Bassett (2009), Bullmore (2009), Cabral (2011) and Bassett (2006), all incorporated by reference. It has been established in the art that the optimal brain network organization is a so called small-world network, like it has been observed for many other real-life networks. Apparently, brain connectivity is not random, but optimally organized. Brain network organization has been linked to cognition [van den Heuvel (2009); Lange (2009); both herein incorporated by reference], and functional network studies have demonstrated that the optimal small-world network is disrupted in AD patients compared to controls and is reconfigured in a disorganised random topology. These disorganizations in the brain network organization can be monitored using conventional imaging techniques such as EEG, MEG and fMRI. There is a range of neurodegenerative disorders characterized by decreased functional connectivity, synapse loss and impaired brain network organization. Alzheimer's disease is such a neurodegenerative disorder and the leading cause of dementia, wherein synapse loss is the strongest structural correlate with cognitive impairment. The main pathological hallmarks of Alzheimer's Disease (AD) include the accumulation of beta-amyloid plaques and neurofibrillary tangles due to abnormal protein processing. From the very start of the disease process, before the disease is diagnosed, there is synaptic loss and reduced synaptic activity and connectivity in specific brain areas, and a deteriorating brain network organization. This results in the classic clinical features of AD: memory impairment, language deterioration, and executive and visuospatial dysfunction. Deteriorating brain network organization and synapse loss are considered to be the most direct correlation to cognitive performance in AD, even more than the number of plaques or tangles, or degree of neuronal loss. As said here above, the link between brain network organization, particularly changes therein, and cognitive function or intelligence has been addressed in the art. Therefore, it is believed that improving synaptic maintenance and preserving or maintaining brain network organization may well be a primary therapeutic target in AD.

During the last decennium, uridine, choline and omega-3 fatty acids such as DHA have attracted attention as active components in treating the ‘AD associated functional symptoms’ such as cognitive dysfunction and age-associated memory impairment (AAMI), see e.g. WO2007/089703 (Massachusetts Institute of Technology) and WO 2009/002165 (N.V. Nutricia). In accordance therewith, improved memory performance by the intake of medical nutrition containing a combination of specific nutrients DHA/EPA, UMP, choline, phospholipids and vitamins B, C and E and selenium has been demonstrated in drug-naive mild AD patients in independent randomized controlled trials (see e.g. Scheltens et al., “Efficacy of a medical food in mild Alzheimer's disease: A randomized controlled trial” Alzheimer's & Dementia 6 (2010), 1-10). A link between intervention and synaptic formation in early AD was suggested.

In the art there is a need for improving and/or supporting the synaptic function, particularly targeting functional brain connectivity and/or a need for preserving brain network organization, in order to treat neurological disorders, in particular CNS disorders, and preferably neurodegenerative disorders, such as AD, possibly still in pre-clinical disease stages.

### SUMMARY OF THE INVENTION

The inventors have observed that after administration of a product comprising (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof, (impaired) functional synaptic connectivity in a subject's brain can be improved and/or preserved and brain network organization preserved, in particular in a subject suffering from or at (high or increased) risk of a neurodegenerative disorder which are characterized by compromised functional connectivity and/or compromised brain network organization, particularly Alzheimer's disease.

In the art, functional connectivity reflects the correlations between spatially remote neurophysiological events, thus characterizing functional interactions in the brain. In the context of the invention, the terminology ‘functional connectivity’, ‘functional brain connectivity’ and ‘functional synaptic connectivity’ are used interchangeably, and refer to the concept of statistical interdependencies between signals of brain activity as a tentative index of functional interactions. This definition is taken from Stam et al. Hum Brain Map 28 (2007) 1178-93. As laid down in the background description already, there is a fundamental distinction between functional connectivity (temporal correlations between remote neurophysiological events) and effective connectivity (the influence one neural system exerts one another), and functional connectivity is also different from structural or anatomic connectivity dealing with the physical or structural synaptic connections. An overview is provided by Friston Human Brain Mapping 2: 56-78 (1994), its contents herein incorporated by reference. In the field of neuroimaging, functional connectivity is a well-known and distinct concept.

The concept ‘brain network organization’ is known in the field. In the context of the invention, ‘deteriorated brain network organization’, ‘impaired brain network organization’, ‘compromised brain network organization’ and ‘disorganized brain network organization’ are all used interchangeably throughout the application, and reflect the changes in brain network organization compared to an optimal ‘small-world’ network organization. The brain network organization can be assessed based on measures of functional connectivity, for which so-called graphs can be constructed and analysed, providing insight into the specific organisation, rather than the strength, of the connections. The organisation of such graphs can be quantified using the theoretical framework of graph theory, see for instance van Steen (2010) and Watts (1998), their contents incorporated by reference. While there exist multiple ways to assess brain network organization, a well-established brain network organization characterization also applied in the clinical trials described therein makes use of a ‘clustering coefficient C’, indicating the interconnectedness of neighbouring points or local connectivity, and the ‘path length L’ which indicates global connectivity, integration, or efficiency. A healthy brain network organization is referred to as the small-world network, combining high local connectivity with short path length. The optimal healthy brain has a small-world network index [SWI] represented by a high clustering coefficient and a low characteristic path length. More details are provided in FIG. 1, discussed further below.

The inventors were the first to identify that impaired functional connectivity and/or impaired brain network organization in a subject could be advantageously affected by administering the above-defined composition, using electroencephalography (EEG) for monitoring changes in brain function. The results are discussed in more detail further below. With these insights, therapies associated with impaired functional connectivity and/or impaired brain network organization, such as AD, could be developed more effectively.

### LIST OF PREFERRED EMBODIMENTS

1. Use of a composition for the manufacture of a product for improving or preserving functional brain connectivity and/or functional synaptic activity and/or brain network organization in a subject in need thereof, and/or slowing down, preventing or reversing impaired functional brain connectivity and/or impaired functional synaptic activity and/or impaired brain network organization in a subject in need thereof, wherein said composition comprises:
   i) one or more of uridine and cytidine, or salts, phosphates, acyl derivatives or esters thereof; and
   ii) a lipid fraction comprising at least one of docosahexaenoic acid (22:6; DHA), eicosapentaenoic acid (20:5; EPA) and docosapentaenoic acid (22:5; DPA), or esters thereof
2. Use of a composition for the manufacture of a product for treating a subject in need thereof, wherein said composition comprises:
   i) one or more of uridine and cytidine, or salts, phosphates, acyl derivatives or esters thereof; and
   ii) a lipid fraction comprising at least one of docosahexaenoic acid (22:6; DHA), eicosapentaenoic acid (20:5; EPA) and docosapentaenoic acid (22:5; DPA), or esters thereof,
      and wherein said subject is subjected to an imaging technique for assessing or monitoring functional brain connectivity and/or brain network organization.
3. A method for improving or preserving functional connectivity and/or brain network organization in a subject in need thereof, wherein said method comprises administering to said subject a composition comprising:
   i) one or more of uridine and cytidine, or salts, phosphates, acyl derivatives or esters thereof; and
   ii) a lipid fraction comprising at least one of docosahexaenoic acid (22:6; DHA), eicosapentaenoic acid (20:5; EPA) and docosapentaenoic acid (22:5; DPA), or esters thereof,
      and wherein said subject is optionally subjected to an imaging technique for assessing or monitoring functional brain connectivity and/or brain network organization.
4. Use or method according to embodiment 2 or 3, wherein said imaging technique comprises electroencephalography (EEG), functional magnetic resonance imaging (fMRI) and/or magnetoencephalography (MEG).
5. Use or method according to any one of the preceding embodiments, wherein said subject suffers from a neurological disorder, in particular a neurocognitive disorder, neurodevelopmental disorder or a depressive disorder, more preferably a neurocognitive disorder selected from the group consisting of Alzheimer's disease, Mild Cognitive impairment (MCI), Parkinson's Disease, and Huntington's Disease, or a neurodevelopmental disorder selected from the group consisting of Attention Deficit/Hyperactivity Disorder and Autism Spectrum Disorder, or a depressive disorder selected from the group consisting of depression and Chronic Depressive Disorder.
6. Use or method according to embodiment 5, wherein said subject suffers from or is at risk of a memory or cognitive disorder, memory decline or cognitive dysfunction, such as Age Associated Memory Impairment (AAMI), Alzheimer's Disease, multiple sclerosis, vascular dementia, frontotemporal dementia, semantic dementia or dementia with Lewy bodies.
7. Use or method according to embodiment 5, wherein said subject suffers from or is at risk of AD, dementias, MCI, memory disorders, Parkinson, obsessive compulsive disorder, Tourette's syndrome, depression, schizophrenia, Autism Spectrum Disorders (ASD), post traumatic stress syndrome (PTSD), traumatic brain injury, PKU, alcoholism, Down syndrome, epilepsy, ALS, HIV, bipolar disorder, Multiple Sclerosis, Huntington, attention-deficit/hyperactivity disorder, and autism (asperger).
8. Use or method according to any one of the preceding embodiments, wherein said subject suffers from or is at risk of Alzheimer's Disease or dementia syndrome, including mild or prodromal AD or dementia.
9. The method according to embodiment 8, wherein said neurodegenerative disorder is AD or dementia syndrome.
10. Use or method according to any one of the preceding embodiments, wherein said composition comprises choline, or salts or esters thereof, preferably 200-600 mg choline per daily dose or per 100 ml composition.
11. Use or method according to any one of the preceding embodiments, wherein said composition comprises at least one, preferably at least two, most preferably all B vitamins selected from the group consisting of vitamin B6, vitamin B12 and vitamin B9.
12. Use or method according to any one of the preceding embodiments, wherein said composition comprises, per daily dose or preferably per 100 ml composition, at least 500 mg of DHA, preferably at least 600 mg of DHA, and at least 50 mg of uridine, preferably at least 100 mg of uridine.
13. Use or method according to any one of the preceding embodiments, wherein the composition comprises, per daily dose or preferably per 100 ml composition:
   50-1000 mg phospholipids,
   0.5-3 mg vitamin B6,
   50-500 μg folic acid,
   1-30 μg vitamin B12.
14. Use or method according to any one of the preceding embodiments, wherein the composition comprises, per daily dose or preferably per 100 ml composition:
   100-500 mg, preferably 200-400 mg EPA,
   1000-1500 mg, preferably 1100-1300 mg DHA,
   50-600 mg, preferably 60-200 mg phospholipids,
   200-600 mg, preferably 300-500 mg choline,
   400-800 mg, preferably 500-700 mg UMP (uridine monophosphate),
   20-60 mg, preferably 30-50 mg vitamin E (alpha-TE),
   60-100 mg, preferably 70-90 mg vitamin C,
   40-80 μg, preferably 50-70 μg selenium,
   1-5 μg, preferably 2-4 μg vitamin B12,
   0.5-3 mg, preferably 0.5-2 mg vitamin B6, and
   200-600 μg, preferably 300-500 μg folic acid.
15. Use of EEG, fMRI and/or MEG for monitoring functional synaptic connectivity and/or synaptic function and/or brain network organization in intervention studies wherein a subject in need thereof is administered a composition comprising:
   i) one or more of uridine and cytidine, or salts, phosphates, acyl derivatives or esters thereof; and
   ii) a lipid fraction comprising at least one of docosahexaenoic acid (22:6; DHA), eicosapentaenoic acid (20:5; EPA) and docosapentaenoic acid (22:5; DPA), or esters thereof.
16. A composition for use in improving or preserving functional brain connectivity and/or brain network organization in a subject in need thereof, wherein said composition comprises:
   i) one or more of uridine and cytidine, or salts, phosphates, acyl derivatives or esters thereof; and
   ii) a lipid fraction comprising at least one of docosahexaenoic acid (22:6; DHA), eicosapentaenoic acid (20:5; EPA) and docosapentaenoic acid (22:5; DPA), or esters thereof.
17. The composition according to embodiment 16, wherein said subject is subjected to an imaging technique for assessing or monitoring functional brain connectivity and/or brain network organization.

### LIST OF FIGURES

FIG. 1 is a schematic representation of a network model based on clustering coefficient C and path length L. Left: ordered model with high C and high L, middle: small-world model with high C and low L; right: random model with low C and low L. Source: Watts and Strogatz, Nature (1998);

FIG. 2 shows the different stages of cognitive decay in Alzheimer's Disease. Source: Sperling et al. Toward defining the preclinical stages of Alzheimer's disease: recommendations from the National Institute on Aging and the Alzheimer's Association workgroup. Alzheimers Dement (2011);

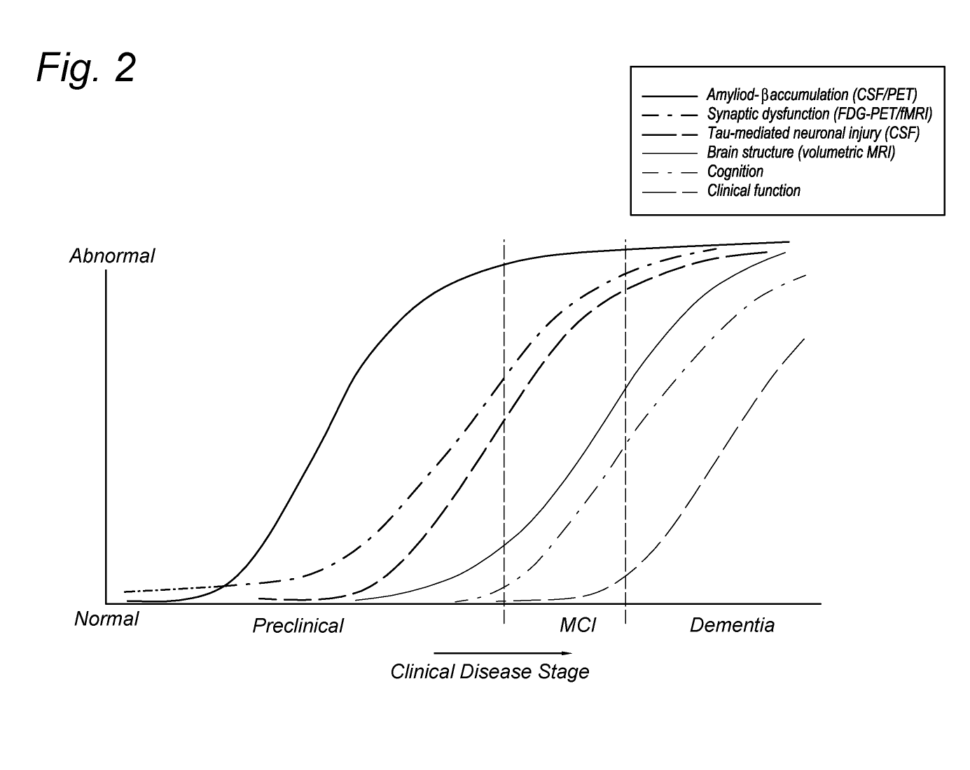

FIG. 3 shows peak frequency (FIG. 3A) and PLI (FIG. 3B) in a 24 weeks intervention study using the composition (triangles; ‘active’) according to the invention. It shows that peak frequency (indicative of brain activity) slowed in the control group and remained relatively stable in the active group (p=0.019). In addition, functional connectivity analysis (PLI) revealed a significant intervention effect in favor of the active group over 24 weeks (p=0.011). This parameter corresponds to functional brain connectivity according to the invention;

FIG. 4 depicts the mean normalized clustering coefficient (FIG. 4a) and mean normalized path length (FIG. 4b) in the beta band in a 24 weeks intervention study. It shows that both network parameters remained stable in the active group and decreased in the control group, which were significantly different between the groups (p=0.009 and p=0.0053 for normalized clustering coefficient and normalized path length, respectively). For clarification: C=clustering coefficient, gamma=normalized clustering coefficient; L=path length, lambda=normalized path length.

### DETAILED DESCRIPTION OF THE INVENTION

In one aspect, the invention pertains to the use of a composition (for the manufacture of a product) for use in:
improving or preserving functional brain connectivity and/or functional synaptic activity in a subject in need thereof, and/or
slowing down, preventing or reversing impaired functional brain connectivity and/or impaired functional synaptic activity; and/or
preserving and/or improving brain network organization in a subject in need thereof; and/or
slowing down or preventing deteriorating brain network organization, wherein said composition comprises:
i) one or more of uridine and cytidine, or salts, phosphates, acyl derivatives or esters thereof; and
ii) a lipid fraction comprising at least one of docosahexaenoic acid (22:6; DHA), eicosapentaenoic acid (20:5; EPA) and docosapentaenoic acid (22:5; DPA), or esters thereof.

In particular, the invention relates to the use of a composition (for the manufacture of a product) for use in improving or preserving functional (synaptic) connectivity and/or preserving brain network organization, wherein said composition comprises:
i) one or more of uridine and cytidine, or salts, phosphates, acyl derivatives or esters thereof; and
ii) a lipid fraction comprising at least one of docosahexaenoic acid (22:6; DHA), eicosapentaenoic acid (20:5; EPA) and docosapentaenoic acid (22:5; DPA), or esters thereof.

In a preferred embodiment, the composition further comprises iii) choline, or salts or esters thereof.

The inventors' contribution is based on an intervention study using EEG for monitoring functional brain connectivity and functional connectivity networks (by applying graph theoretical analysis on the EEG data. It is noticed that EEG is one of the biomarkers suitable for monitoring functional brain connectivity and functional brain networks in neurodegenerative pathology. Other suitable direct imaging techniques available to either measure brain function or derivatives thereof (such as blood flow or metabolism) are magnetoencephalography (MEG), functional magnetic resonance imaging (fMRI), fluorodeoxy glucose positron emission tomography (FDG-PET), near infra-red spectroscopy (NIRS), single-photon emission computed tomography (SPECT), arterial spin labeling (ASL) This is a non-exhaustive list of (potential) functional brain connectivity monitoring techniques, all useful in the context of the present invention.

Functional MRI (fMRI) enables the visualization of regional cerebral blood flow and blood oxygenation, indicating areas of increased or decreased neural activity (Sorg et al. Curr. Alzheimer Res. 6 (2009) 541-553). In FDG-PET glucose metabolism rates in brain are visualized, based on the fact that the demand for glucose is driven by synaptic terminals which generate ATP needed for synthesis, release, and recycling of neurotransmitter molecules, the maintenance of the normal resting potential and the recovery from action potentials. The cerebral metabolic rate of glucose as measured with FDG-PET, sometimes referred to as ‘metabolic connectivity’, is a direct index of synaptic functioning (see e.g. Mosconi. et al. Ann. N.Y. Acad. Sci. 1147 (2008) 180-195). The contents of all references cited in this paragraph are herein considered incorporated by reference.

Here, EEG and MEG are particularly preferred for assessing functional connectivity and functional connectivity networks, since both directly measure neuronal activity. MEG is a technique that allows activity in the brain to be mapped by analyzing localized fluctuations in a magnetic field caused by neuronal currents. In line with MEG, EEG records electrical activity on the scalp as an indicator of functional connectivity. Like MEG, EEG can be used to identify progression of neurodegenerative disorders such as AD based upon the characteristic ‘slowing’ of the EEG (MEG) pattern, and provides indirectly valuable information on synaptic function and connectivity. The EEG electrodes can be regarded as the nodes of a brain network with the synchronization strength or probability between the nodes being the connections between the points. This is based on the notion that brain areas that are connected will synchronize their activity.

A link between EEG and MEG with brain networks in AD patients—compared to healthy individuals—is explained in Stam (2009) and Stam (2010), their contents is herein incorporated by reference. It was observed that AD patients, amongst others, displayed an increase in slow frequency bands (delta, theta) and a decrease in fast frequency bands (alpha, beta); and slowing of peak frequency, when compared to the normal control group. All these parameters reflect underlying brain activity/oscillations.

Brain network organization is typically studied using EEG, MEG or fMRI. Here, EEG has been used, but similar results could be likewise be retrieved using MEG or fMRI, the latter having the advantage of a higher spatial resolution. The amount of synchronization in the EEG signals of different brain regions can be established with synchronization measures. Based on the pairwise synchronization values, the networks can be quantified with network analysis. Complex brain networks have been characterized with graph theory (see Stam (2009)), for example based on a clustering coefficient C and a characteristic path length L. The clustering coefficient is a measure of the local ‘interconnectedness’ of the graph, whereas the path length is believed to be an indicator of its overall connectedness. According to Watts (1998), graphs with many local connections and a few random long distance connections are characterized by a high cluster coefficient and a short path length; such near-optimal networks are designated as “small-world” networks, indicated with the small world index (SWI). A small world-like network architecture may be optimal for synchronizing neural activity between different brain regions. While healthy subjects have brain networks with small-world topology, characterized by combination of high clustering and short path lengths, patients suffering from impaired functional brain connectivity exhibit more random brain networks due to loss of critical communication lines. These patients show a loss of the optimal brain organization, which is believed to indicate loss of synaptic connections and disrupted neuronal communication.

In the present intervention study underlying the invention, EEG showed that the peak frequency in a group of AD patients having a MMSE score of 20 or higher was stabilized upon administration of the composition according to the invention over a first period of 12 weeks, and even increased in the subsequent 12 weeks of the 24-week study, where the same peak frequency in the control group continued to decrease over the complete length of the study. More details are provided in the detailed description and the experimental section below.

In a further aspect, the invention pertains to the use of a composition comprising (i)-(ii) and optionally (iii) as defined above in the manufacture of a product for treating a subject in need thereof, and subjecting said subject to an imaging technique for assessing functional connectivity, preferably one or more imaging techniques selected from the list consisting of electroencephalography (EEG), magnetoencephalography (MEG), functional magnetic resonance imaging (fMRI), fluorodeoxy glucose positron emission tomography (FDG-PET), near infra-red spectroscopy (NIRS), single-photon emission computed tomography (SPECT), arterial spin labeling (ASL), preferably EEG and/or MEG. It is preferred assessing the subject for functional synaptic connectivity, synaptic activity, synaptic function and/or synchronous activity of synapses, all associated with functional brain connectivity rather than anatomical or effective connectivity. In a preferred embodiment, the imaging technique is selected from EEG, MEG and fMRI. The composition is preferably administered to said subject at least on daily basis, preferably for at least 12 weeks. It is preferred to apply graph theoretical analysis to the functional connectivity results of the imaging technique, yielding information on the organization of functional connectivity networks. For example, a clustering coefficient C and a characteristic path length L may be calculated. In a further step, the SWI may be calculated from those parameters.

The method preferably involves monitoring said subject using EEG. In one aspect, the EEG involves at least monitoring for shifts in Phase Lag Index (PLI). Advanced methods for the analysis of EEG and MEG signals, such as quantitative frequency analysis and analysis of functional connectivity, show an increase of relative power of activity in the lower frequency bands (delta and theta band), a decrease in relative power in the higher frequency bands (alpha and beta bands), slowing of the peak frequency, and a decreased functional connectivity between brain regions in AD patients compared to controls. The Phase Lag Index (PLI) in these frequency bands is a good indicator of brain synchronization and brain functional connectivity. Another indicator is synchronization likelihood. PLI and synchronization likelihood can be used together or independently to monitor the progress of AD or dementia, particularly functional connectivity. More details are provided in Brenner et al. (1998) and De Haan et al. (2008, 2009), their contents herewith considered incorporated by reference.

In one aspect, the invention pertains to a method for monitoring the effect of a composition for treating a subject suffering from compromised or decreased functional connectivity, said subject preferably suffering from (or at risk of) AD, wherein said method involves measuring or observing Phase Lag Index (PLI).

In one aspect, the invention pertains to a method for monitoring the effect of a composition for treating a subject suffering from compromised or decreased brain network organization, said subject preferably suffering from (or at risk of) AD, wherein said method involves determining clustering coefficient C and characteristic path length L, and optionally calculating the SWI from those parameters. A suitable tool for arriving at these parameters is graph theory analysis. The measurements could be carried out using EEG, MEG or fMRI.

In one aspect, the invention pertains to a method for monitoring the effect of a composition for treating a subject suffering or at risk of a neurodegenerative disorder and/or compromised or decreased functional connectivity and/or compromised brain network organization, said subject preferably suffering from (or at risk of) AD, wherein said method involves measuring or observing (changes or shifts in) Phase Lag Index (PLI). In one aspect, the invention pertains to a method for monitoring the effect of a composition, preferably a composition comprising the aforementioned ingredients, and as further outlined below, for treating an elderly subject or a subject suffering or at risk of a neurodegenerative or neurological disorder, preferably a CNS disorder, preferably a disorder associated with cognitive impairment and/or compromised or decreased functional connectivity and/or compromised brain network organization, wherein said method involves determining clustering coefficient C, characteristic path length L, and optionally SWI.

The method or use of the invention comprises administering the composition comprising the aforementioned ingredients, and as further outlined below, to a subject in need thereof. The prophylactic or preventive aspect includes reducing the risk of occurring of the disorders.

The treatment preferably involves daily administration of the product, preferably for at least 12 weeks. The product is preferably administered (daily) for at least 13 weeks, more preferably at least 14, 15, 16, 17, 18, 19, 20, 21, 22, 23, most preferably at least 24 weeks.

### Functional Connectivity

The terms ‘functional synaptic connectivity’, ‘functional brain connectivity’ and ‘functional neuronal connectivity’ are considered interchangeably, abbreviated in the context of the invention as ‘functional connectivity’. With ‘improving or preserving functional (brain) synaptic connectivity’ it is understood that impaired functional synaptic activity, impaired synchronous activity of synapses and/or impaired functional connectivity in the brain areas, associated with various neurological disorders, in particular various CNS (central nervous system) disorders, preferably neurodegenerative disorders, such as AD, is reduced, slowed down, halted or even reversed.

While it is not possible to measure synaptic connectivity directly in humans, neuronal functional connectivity in humans can be studied using an imaging technique for assessing functional connectivity, for instance imaging techniques such as electroencephalography (EEG), magnetoencephalography (MEG), functional magnetic resonance imaging (fMRI), fluorodeoxy glucose positron emission tomography (FDG-PET), near infra-red spectroscopy (NIRS), single-photon emission computed tomography (SPECT), arterial spin labeling (ASL), preferably electroencephalography (EEG) and/or magnetoencephalography (MEG). As explained above, the EEG/MEG signal is a compound of activity of many synapses and is therefore a derivative of underlying synaptic function. Likewise, fMRI is also suited.

### Brain Network Organization

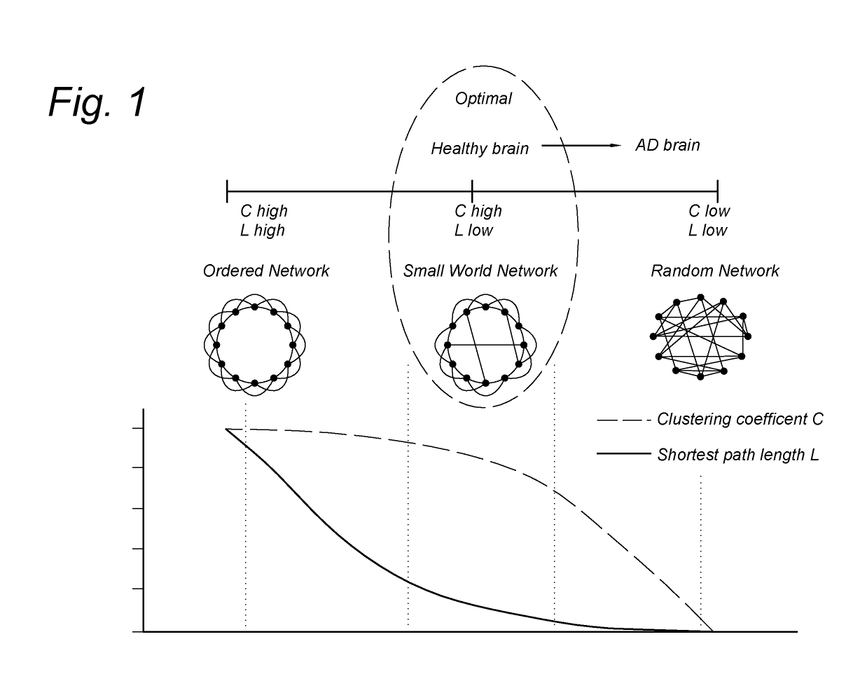

Functional brain network organization can be constructed from measures on functional connectivity. These networks can be assessed with respect to organization using graph theory, providing insight in the specific organization of the connections. It results in a measure for local connectivity and global integration of the network: From the networks, using graph theory, several measures can be computed to characterize the networks, such as the clustering coefficient and the path length. The measure clustering coefficient C indicates the interconnectedness of neighboring points (local connectivity) and has a high value in case of an ordered network (FIG. 1, left) and a low value in case of a random network (FIG. 1, right). The measure path length L is a measure of the ease to traverse the network (global connectivity, integration, or efficiency). Graphs are data structures which have nodes and edges between the nodes. The clustering coefficient of every node is computed as the ratio of the number of connections between its neighbors divided by the maximum possible connections between its neighbors. The clustering coefficient (C) of the network is calculated as the mean of the clustering coefficients of all the nodes in the network. The mean minimum path length of a node is computed as the average of minimum distances from that node to all the remaining nodes in the network. The characteristic path length (L) of the network is the average of the mean minimum path lengths of all the nodes in the network. The clustering coefficient and path length of nodes completely disconnected with the network are set as 0 and ‘infinity’ respectively, and these nodes are thus excluded while computing C and L.

In order to evaluate the network for small-world properties, i.e. determining the SWI, the clustering coefficient and the characteristic path length of the network may be normalized with respect to their corresponding values obtained and averaged across 1000 random networks with the same number of nodes and degree distribution.

In an ordered network, it takes many steps to reach the other side of the network (high path length value, FIG. 1, left) where in a random network it only takes a few steps (low path length value, FIG. 1, right). In between the ordered and random network is the small-world network, combining high local connectivity with short path length (FIG. 1, middle).

At the developed stages of neurodegeneration, the above may manifest through impairment of learning abilities, memory function and/or cognition. However, as addressed above, the loss of functional brain connectivity and deteriorating brain network organization may precede such clinical stages long before, and may now be addressed using the composition of the invention. Preservation and improvement are measured relative to a control group of subjects suffering from the same condition but not given the composition of the invention.

### Subject

In particular, the subject is a human being that suffers from (or is at risk of) decreased/disturbed/impaired functional connectivity, particularly suffering from a neurological disorder, more preferably a CNS disorder. More in particular, these disorders are neurocognitive disorders, neurodevelopmental disorders and depressive disorders, and more preferred these are neurocognitive disorders. The preferred neurocognitive disorders are degenerative neurocognitive disorders, non-degenerative neurocognitive disorders and vascular related neurocognitive disorders, more preferred degenerative neurocognitive disorders. Following classifications used in the art, a representation of those disorders associated with impaired functional brain connectivity and deteriorated brain network organization is listed in table 1. The claimed combination is preferably used for treating and/or preventing (including reducing the risk of occurrence) of any of those disorder categories, disorder subcategories and preferably the disorders listed in table 1.

The preferred degenerative neurocognitive disorders are Alzheimer's disease (Bozzali et al., 2011; Stam, 2010), Mild Cognitive impairment (MCI) {Han, 2011 #8924}, Parkinson's Disease (Stam, 2010) and Huntington's Disease (Wolf et al., 2008), more preferred Alzheimer's disease and Mild Cognitive impairment. The preferred neurodevelopmental disorders are Attention Deficit/Hyperactivity Disorder (Cubillo and Rubia, 2010; Konrad and Eickhoff, 2010) and Autism Spectrum Disorder (Gepner and Feron, 2009), more preferred Attention Deficit/Hyperactivity Disorder. The preferred depressive disorders are depression (Cao et al., 2012) and Chronic Depressive Disorder, more preferred depression. The references cited in this paragraph are specified further below; the contents of those citations is herein incorporated by reference

TABLE 1
disorders associated with impaired functional brain connectivity/
brain network organization

| Disorder | Disorder | |
|---|---|---|
| Category | Subcategory | Disorder |
| Neurocognitive | Degenerative | Alzheimer's Disease |
| Disorder | neurocognitive | Mild Cognitive impairment |
| | disorders | (MCI) |
| | | Parkinson's Disease |
| | | Huntington's Disease |
| | Non-degenerative | Age Associated Memory |
| | neurocognitive | Impairment (AAMI) |
| | disorders | Semantic dementia |
| | Vascular related | Vascular dementia |
| | neurocognitive | |
| | disorders | |
| Neurodevelopmental | | Attention Deficit/Hyperactivity |
| Disorders | | Disorder |
| | | Autism Spectrum Disorder |
| Depressive | | Depression |
| Disorders | | Chronic Depressive Disorder |

More in particular, the subjects are at risk of or are suffering from disorders selected from neurocognitive disorders, neurodevelopmental disorders and depressive disorders, and more preferred neurocognitive disorders. The subject is preferably at risk of or suffering from degenerative neurocognitive disorders, non-degenerative neurocognitive disorders and vascular related neurocognitive disorders, more preferred degenerative neurocognitive disorders.

More particularly the subject is a human being that suffers from (or is at risk of) a memory or cognitive disorder, memory decline or cognitive dysfunction, such as Age Associated Memory Impairment (AAMI), multiple sclerosis, vascular dementia, frontotemporal dementia, semantic dementia or dementia with Lewy bodies, and Alzheimer's Disease, and/or psychiatric and developmental disorders, including obsessive-compulsive disorder, Tourette's syndrome, depression, schizophrenia, attention-deficit/hyperactivity disorder, and autism (asperger). In the aforementioned conditions, memory and cognition functions are known to deteriorate in time. Possibly, the subject does not suffer from any clinical stages associated with impaired functional connectivity and/or deteriorated brain network organization yet.

In particular, the subject can be a human being who has not yet been diagnosed a (specific) disease (such as a neurodegenerative disease, e.g. AD), but has an impaired functional connectivity and/or disturbed brain network organization, as determined and/or measured by any (imaging) technique suitable for assessing functional connectivity and brain network organization.

In a preferred embodiment, the subject is a human being that suffers from (or is at risk of) decreased/disturbed/impaired functional connectivity and/or decreased/disturbed/impaired brain network organization, preferably suffering from AD, dementias, MCI, memory disorders, Parkinson, obsessive compulsive disorder, Tourette's syndrome, depression, schizophrenia, Autism Spectrum Disorders (ASD), post traumatic stress syndrome (PTSD), traumatic brain injury, PKU, alcoholism, Down syndrome, epilepsy, ALS, HIV, bipolar disorder, Multiple Sclerosis, Huntington, attention-deficit/hyperactivity disorder, and autism (asperger), more preferably suffering from AD, dementias, MCI, memory disorders, or Parkinson. The subject possibly does not suffer from any clinical stages associated with impaired functional connectivity and/or impaired brain network organization yet.

In a preferred embodiment, the subject is a human being that suffers from (or is at risk of) decreased/disturbed/impaired functional connectivity and/or decreased/disturbed/impaired brain network organization, preferably suffering from a memory or cognitive disorder, memory decline or cognitive dysfunction. The subject is preferably suffering from cognitive dysfunction associated with Alzheimer's disease [AD], Pick's disease (or frontotemporal dementia, frontal variant), Lewy Body disease, Huntington's disease, or ‘dementia syndrome’. Dementia syndrome encompasses vascular dementia, frontotemporal dementia and semantic dementia. The subject possibly does not suffer from any clinical stages associated with impaired functional connectivity and/or impaired brain network organization yet.

The subject is preferably a human, preferably an elderly human being, preferably at least 50 years of age. The subject is preferably an AD or dementia patient. In one aspect, the invention is concerned with the treatment of persons suffering from Alzheimer's disease, dementia and/or elderly.

In one embodiment, the subject is preferably a drug-naïve subject, which subject has preferably not been administered any drug for memory improvement and or for AD or dementia at least 4 weeks prior to the administration of a composition according to the invention. Preferably, the term ‘drug naïve’ as used in the present invention refers to subjects who do not ingest one or more of cholinesterase inhibitors, N-methyl-D-aspartate (NMDA) antagonists and ginkgo biloba during treatment with the composition of the invention, and preferably have not taken any cognitive ability-affecting drugs in the 4 weeks prior to the treatment.

In one aspect, the subject is a mild cognitive impairment (MCI) patient (or ‘mild AD patient’ or ‘mild dementia patient’) or an AAMI patient. The patient group may also encompass prodromal patients of neurological disorders, in particular prodromal AD patients or drug-naïve prodromal dementia patients. A ‘prodromal dementia patient’ is a person who does not suffer from a senile dementia as defined above, but has an increased likelihood to develop senile dementia. Likewise a ‘prodromal Alzheimer patient’ is a person who does not suffer from AD, but has an increased likelihood to develop AD. The diagnostic tools that are used to classify the patients as prodromal patients are available in the art, and for instance summarized in WO 2009/002164, its contents herein incorporated by reference.

In yet a further way of characterizing the subject may be characterized by having a mini-mental state examination [MMSE] of 20-30. The MMSE is a standardized test developed in the art to distinguish between the various (pre-) stages of dementia. It involves a brief 30-point questionnaire that is used to assess cognition. In the time span of about 10 minutes it samples various functions including memory and orientation. The MMSE test includes simple questions and problems in a number of areas: the time and place of the test, repeating lists of words, language use and comprehension, and basic motor skills. Any score of 27 or higher (out of 30) is interpreted as effectively normal; 20-26 indicates mild dementia; 10-19 moderate dementia, and below 10 severe dementia. Copyrights prevent the inventors from including a copy of the questionnaire into the specification, but it is readily accessible on the internet and available through copyright owner Psychological Assessment Resources (PAR). It is first introduced by Folstein et al. (Psych Res 12:189, 1975), and is widely used with small modifications to assess cognition. Preferably, in the present invention, the subjects have a mini-mental state examination (MMSE) of 20-30, more preferably of 20-26, even more preferably a MMSE of 24, 25 or 26. More preferably, the subject having the aforementioned MMSE score range has (or suffers from) Alzheimer's disease, mild cognitive impairment (MCI), age-associated memory impairment (AAMI), multiple sclerosis, vascular dementia, frontotemporal dementia, semantic dementia or dementia with Lewy bodies.

Most preferably, the subjects as treated in the present invention suffer from mild Alzheimer's disease characterized by a MMSE of 20-26, preferably 24-26. In one embodiment, the subject is drug naïve.

With the inventors' insights it is possible to target functional brain connectivity and brain network organization as the principle cause of clinical stages such as cognitive and memory decline. These are pathological biomarkers that precede the functional abnormalities associated with the aforementioned neurodegenerative conditions. The composition according to the invention is therefore particularly suited for treating neurodegenerative disorders as described above, particularly CNS disorders, more preferably CNS disorders associated with decreased/disturbed/impaired functional connectivity and/or decreased/disturbed/impaired brain network organization, in the early stages, at the onset, particularly in those stages where decline in cognitive abilities is still insignificant or not observed. The new insights offer the opportunity to start intervention already in people that are at increased risk of developing the above disorders years before diagnosis of disease would be diagnosed.

### Product

Throughout the application, the terms ‘product’ and ‘composition’ are used interchangeably and account for the combination of ingredients administered to a subject in need thereof.

In one aspect of the present invention, the composition according to the invention may be used as a pharmaceutical product comprising one or more pharmaceutically acceptable carrier materials.

In another aspect of the present invention, the composition according to the invention may be used as a nutritional product, for example as a nutritional supplement, e.g., as an additive to a normal diet, as a fortifier, to add to a normal diet, or as a complete nutrition.

The pharmaceutical product, preferably for enteral application, may be a solid or liquid galenical formulation. Examples of solid galenical formulations are tablets, capsules (e.g. hard or soft shell gelatine capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as flavoring agents, preservatives, stabilizers, emulsifying agents, buffers and the like may be added in accordance with accepted practices of pharmaceutical compounding. While the individual active ingredients are suitably administered in a single composition, they may also be administered in individual dosage units.

Hence, the invention further relates to a kit of parts comprising i) one or more of uridine and cytidine, or salts, phosphates, acyl derivatives or esters thereof; and ii) a lipid fraction comprising at least one of docosahexaenoic acid (22:6; DHA), eicosapentaenoic acid (20:5; EPA) and docosapentaenoic acid (22:5; DPA), or esters thereof, for the aforementioned use or for use in the aforementioned method. In one embodiment, it is preferred to include iii) choline, or salts or esters thereof.

If the composition is a pharmaceutical product, such product may contain the daily dosage in one or more dosage units. The dosage unit may be in a liquid form or in a solid form, wherein in the latter case the daily dosage may be provided by one or more solid dosage units, e.g. in one or more capsules or tablets.

In another aspect of the present invention, the composition according to the invention may be used in a nutritional product comprising at least one component selected from the group of fats, proteins, and carbohydrates. It is understood that a nutritional product differs from a pharmaceutical product by the presence of nutrients which provide nutrition to the subject to which the composition is administered, in particular the presence of protein, fat, digestible carbohydrates and dietary fibers. It may further contain ingredients such as minerals, vitamins, organic acids, and flavoring agents. Although the term “nutraceutical product” is often used in literature, it denotes a nutritional product with a pharmaceutical component or pharmaceutical purpose. Hence, the nutritional composition according to the invention may also be used in a nutraceutical product.

The product of the invention is an enteral composition, intended for oral administration. It is preferably administered in liquid form. In one embodiment, the product comprises a lipid fraction and at least one of carbohydrates and proteins, wherein the lipid composition provides between 20 and 50 energy % of the food product. In one embodiment, the food product is a liquid composition containing between 0.8 and 1.4 kcal per ml.

Preferably, the composition comprising (i) and (ii) further comprises choline.

Preferably the composition comprising (i) and (ii) further comprises one or more of: phospholipids, vitamin E, vitamin C, selenium, vitamin B12, vitamin B6 and folic acid.

More preferably the composition comprises DHA, EPA, a uridine source (preferably UMP), phospholipids, choline, vitamin E, vitamin C, selenium, vitamin B12, vitamin B6 and folic acid.

### DHA/EPA

The composition comprises at least one ω-3 polyunsaturated fatty acid (LC PUFA; having a chain length of 18 and more carbon atoms) selected from the group consisting of docosahexaenoic acid (22:6; DHA), eicosapentaenoic acid (20:5; EPA) and docosapentaenoic acid (22:5 ω-3; DPA), preferably at least one of DHA and EPA. Preferably the present composition contains at least DHA, more preferably DHA and EPA. EPA is converted to DPA (ω-3), increasing subsequent conversion of DPA to DHA in the brain. Hence, the present composition preferably contains a significant amount of EPA, so to further stimulate in vivo DHA formation.

The DHA, EPA and/or DPA are preferably provided as triglycerides, diglycerides, monoglycerides, free fatty acids or their salts or esters, phospholipids, lysophospholipids, glycerol ethers, lipoproteins, ceramides, glycolipids or combinations thereof. Preferably, the present composition comprises at least DHA in triglyceride form.

In terms of daily dosage, the present method preferably comprises the administration of 400 to 5000 mg DHA+EPA+DPA (preferably DHA+EPA) per day, more preferably 500 to 3000 mg (preferably DHA+EPA) per day, most preferably 1000 to 2500 mg (preferably DHA+EPA) per day. DHA is preferably administered in an amount of 300 to 4000 mg per day, more preferably 500 to 2500 mg per day.

The present composition preferably comprises 1-40 wt. % DHA based on total fatty acids, preferably 3-36 wt. % DHA based on total fatty acids, more preferably 10-30 wt. % DHA based on total fatty acids. The present composition preferably comprises 0.5-20 wt. % EPA based on total fatty acids, preferably 2-10 wt. % EPA based on total fatty acids, more preferably 5-10 wt. % EPA based on total fatty acids. The above-mentioned amounts take into account and optimize several aspects, including taste (e.g. too high LCP levels reduce taste, resulting in a reduced compliance).

The present composition preferably contains at least one oil selected from fish oil, algae oil and eggs lipids. Preferably the present composition contains fish oil comprising DHA and EPA.

The ratio of the weights of DHA to EPA is preferably larger than 1, more preferably 2:1 to 10:1, more preferably 3:1 to 8:1. The above-mentioned ratios and amounts take into account and optimize several aspects, including taste (too high LCP levels reduce taste, resulting in a reduced compliance), balance between DHA and precursors thereof to ensure optimal effectiveness while maintaining low-volume formulations.

Sources of DHA possible sources of DHA: tuna oil, (other) fish oils, DHA rich alkyl esters, algae oil, egg yolk, or phospholipids enriched with n-3 LCPUFA e.g. phosphatidylserine-DHA.

The present composition preferably contains a very low amount of arachidonic acid (AA). Preferably the weight ratio DHA/AA in the present composition is at least 5, preferably at least 10, more preferably at least 15, preferably up to e.g. 30 or even up to 60. The present method preferably comprises the administration of a composition comprising less than 5 wt. % arachidonic acid based on total fatty acids, more preferably below 2.5 wt. %, e.g. down to 0.5 wt %.

### ALA/LA

It is preferred that the alpha-linolenic acid [ALA] content of the composition is maintained at low levels. The ALA concentration may preferably be maintained at levels less than 2.0 weight %, more preferably below 1.5 weight %, particularly below 1.0 weight %, calculated on the weight of all fatty acids.

Linoleic acid [LA] concentrations can be maintained at normal levels, i.e. between 20 to 30 weight %, although in one embodiment the LA concentration is also significantly reduced to an amount of <15 g/100 g fatty acids and even less than 10 weight %. The LA concentrations are preferably at least 1 weight % of the fatty acids.

The weight ratio omega-6/omega-3 fatty acids in the present product is preferably below 0.5, more preferably below 0.2, e.g. down to 0.05 or to 0.01. The ratio ω-6/ω-3 fatty acids (C 20 and higher) in the present product is preferably below 0.3, more preferably below 0.15, e.g. down to 0.06 or to 0.03.

### MCT

In one embodiment, the composition contains less than 5 weight %, preferably less than 2 weight % of fatty acids of less than 14 carbon atoms.

Medium chain fatty acids [MCT] are defined to be linear or branched saturated carboxylic acids having six (C6:0), seven (C7:0), eight (C8:0), nine (C9:0) or ten (C10:0) carbon atoms. The amount of MCTs are preferably lower than 2 weight %, more preferably lower than 1.5 weight %, most preferably lower than 1.0 weight % of the total fatty acids. In one embodiment, the sum of the medium chain fatty acids C6:0+C7:0+C8:0 over the sum of C9:0 and C10:0 is less than 2:1, more preferably less than 1.8:1, most preferably less than 1.6:1.

### Saturated and Monounsaturated Fatty Acids

The present composition preferably comprises saturated and/or mono-unsaturated fatty acids. The amount of saturated fatty acids is preferably 6-60 wt. % based on total fatty acids, preferably 12-40 wt. %, more preferably 20-40 wt. % based on total fatty acids. In particular the amount of C14:0 (myristic acid)+C16:0 (palmitic acid) is preferably 5-50 wt. %, preferably 8-36 wt. %, more preferably 15-30 wt. %, based on total fatty acids. The total amount of monounsaturated fatty acids, such as oleic acid and palmitoleic acid, is preferably between 5 and 40 wt. %, more preferably between 15 and 30 wt. %. A composition with these preferred amounts was found to be very effective.

### Uridine, UMP

The present composition comprises uridine, cytidine and/or an equivalent thereof, including salts, phosphates, acyl derivatives and/or esters. In terms of uridine, the composition preferably comprises at least one uridine or an equivalent thereof selected from the group consisting of uridine (i.e. ribosyl uracil), deoxyuridine (deoxyribosyl uracil), uridine phosphates (UMP, dUMP, UDP, UTP), nucleobase uracil and acylated uridine derivatives. In one embodiment, cytidine, CMP, citicoline (CDP-choline) may also be applied. Preferably, the composition to be administered according to the present invention comprises a source of uridine selected from the group consisting of uridine, deoxyuridine, uridine phosphates, uracil, and acylated uridine, and cytidine, more preferably selected from the group consisting of uridine, deoxyuridine, uridine phosphates, uracil, and acylated uridine.

Preferably, the present composition comprises an uridine phosphate selected from the group consisting of uridine monophosphate (UMP), uridine diphosphate (UDP) and uridine triphosphate (UTP); and/or a cytidine phosphate (CMP, CDP, CTP, preferably CMP). Most preferably the present composition comprises UMP, as UMP is most efficiently being taken up by the body. Preferably at least 50 weight % of the uridine in the present composition is provided by UMP, more preferably at least 75 weight %, most preferably at least 95 weight %. Doses that must be administered are given as UMP. The amount of uracil sources can be calculated taking the molar equivalent to the UMP amount (molecular weight 324 Dalton).

The present method preferably comprises the administration of uridine (the cumulative amount of uridine, deoxyuridine, uridine phosphates, nucleobase uracil and acylated uridine derivatives) in an amount of in an amount of 0.08-3 g per day, preferably 0.1-2 g per day, more preferably 0.2-1 g per day. The present method preferably comprises the administration of a composition comprising uridine in an amount of 0.08-3 g UMP per 100 ml liquid product, preferably 0.1-2 g UMP per 100 ml liquid product, more preferably 0.2-1 g per 100 ml liquid product. Preferably 1-37.5 mg UMP per kilogram body weight is administered per day. The above amounts also account for any amounts of cytidine, cytidine phosphates and citicoline incorporated in the composition or method.

Preferably, the present composition comprises uridine phosphate, preferably uridine monophosphate (UMP). The UMP is very efficiently taken up by the body. Hence, inclusion of UMP in the present composition enables a high effectivity at the lowest dosage and/or the administration of a low volume to the subject.

### Choline

In a preferred embodiment, the present composition contains choline, a choline salt and/or choline ester. For the remainder of the paragraph, the term ‘choline’ shall be considered to encompass all these equivalents. The choline salt is preferably selected from choline chloride, choline bitartrate, or choline stearate. The choline ester is preferably selected from a phosphatidylcholine and lyso-phosphatidylcholine. The present method preferably comprises the administration of more than 50 mg choline per day, preferably 80 to 2000 mg choline per day, more preferably 120 to 1000 mg choline per day, most preferably 150 to 600 mg choline per day. The present composition preferably comprises 50 mg to 3000 gram choline per 100 ml of the liquid composition, preferably 200 mg to 1000 mg choline per 100 ml. The above numbers are based on choline, the amounts of choline equivalents or sources can be calculated taking the molar equivalent to choline into account.

### Phospholipids

Preferably, the present composition preferably comprises phospholipids, preferably 0.1-50 wt. % phospholipids based on total weight of lipids, more preferably 0.5-20 wt. %, more preferably between 1 and 10% wt. %, most preferably between 1 and 5 wt. % based on total weight of lipids. The total amount of lipids is preferably between 10 and 30 wt. % on dry matter, and/or between 2 and 10 g lipid per 100 ml for a liquid composition. The composition preferably comprises between 0.01 and 1 gram lecithin per 100 ml, more preferably between 0.05 and 0.5 gram lecithin per 100 ml. A composition with these preferred amounts was found to be very effective. In one embodiment, the phospholipids comprise at least two phospholipids selected from the group consisting of phosphatidylcholine, phosphatidylethanolamine, phosphatidylinositol and phosphatidylserine, preferably at least PC and PE.

### Vitamins

The present combination preferably comprises at least one B complex vitamin. The vitamin B is selected from the group of vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid or folate), and vitamin B 12 (various cobalamins). Functional equivalents are encompassed within these terms.

Preferably, at least one vitamin B is selected from the group of vitamin B6, vitamin B12 and vitamin B9. Preferably the present composition comprises at least two selected from the group consisting of vitamin B6, vitamin B12 and vitamin B9. In particular, good results have been achieved with a combination comprising vitamin B6, vitamin B 12 and vitamin B9. Again, functional equivalents are encompassed within these terms.

The vitamin B is to be administered in an effective dose, which dose depends on the type of vitamin B used. As a rule of thumb, a suitable minimum or a maximum dose may be chosen based on known dietary recommendations, for instance as recommended by Institute of Medicine (TOM) of the U.S. National Academy of Sciences or by Scientific Committee on Food (a scientific committee of the EU), the information disclosed herein and optionally a limited amount of routine testing. A minimum dose may be based on the estimated average requirement (EAR), although a lower dose may already be effective. A maximum dose preferably does not exceed the tolerable upper intake levels (UL), as recommended by IOM.

If present in the nutritional composition or medicament, the vitamin B6 is usually present in an amount to provide a daily dosage in the range of 0.1 to 100 mg, in particular in the range of 0.5 to 25 mg, more in particular in the range of 0.5 to 5 mg. The present composition preferably comprises 0.1 to 100 mg vitamin B6 per 100 g (liquid) product, more preferably 0.5 to 5 mg vitamin B6 per 100 g (liquid) product, more preferably 0.5 to 5 mg vitamin B6 per 100 g (liquid) product.

If present in the nutritional composition or medicament, the vitamin B12 is usually present in an amount to provide a daily dosage in the range of 0.5 to 15 μg, in particular in the range of 1 to 10 μg, more in particular in the range of 1.5 to 5 μg. The present composition preferably comprises 0.5-15 μg vitamin B12 per 100 g (liquid) product, more preferably 1 to 10 μg vitamin B12 per 100 g (liquid) product, more preferably 1.5 to 5 μg vitamin B12 per 100 g (liquid) product. The term “vitamin B12” incorporates all cobalbumin equivalents known in the art.

Throughout the application, the terms ‘folic acid’, ‘folate’ and ‘B9’ are used interchangeably. If present in the nutritional composition or medicament, the vitamin B9 is usually present in an amount to provide a daily dosage in the range of 50 to 1000 μg, in particular in the range of 150 to 750 μg, more in particular in the range of 200 to 500 μg. The present composition preferably comprises 50 to 1000 μg folic acid per 100 g (liquid) product, more preferably 150 to 750 μg folic acid per 100 g (liquid) product, more preferably 200 to 500 μg folic acid per 100 g (liquid) product. Folates include folic acid, folinic acid, methylated, methenylated and formylated forms of folates, their salts or esters, as well as their derivatives with one or more glutamic acid, and all in either reduced or oxidized form.

### Vitamins C, E

Vitamin C, or a functional equivalent thereof, may be present in an amount to provide a daily dosage in the range of 20 to 2000 mg, in particular in the range of 30 to 500 mg, more in particular in the range of 75 to 150 mg. In one embodiment, vitamin C, or a functional equivalent thereof, is present in an amount in the range of 20 to 2000 mg, in particular in the range of 30 to 500 mg, more in particular in the range of 75 to 150 mg per 100 ml of the composition.

Tocopherol and/or an equivalent thereof (i.e. a compound having vitamin E activity) may be present in an amount to provide a daily dosage in the range of 10 to 300 mg, in particular in the range of 30 to 200 mg, more in particular in the range of 35 to 100 mg, to prevent oxidative damage resulting from dietary PUFA. In one embodiment, tocopherol and/or equivalent is present in an amount in the range of 10 to 300 mg, in particular in the range of 30 to 200 mg, more in particular in the range of 35 to 100 mg per 100 ml of the composition. The term “tocopherol and/or an equivalent thereof”, and ‘alpha-TE’, as used in this description, comprises tocopherols, tocotrienols, pharmaceutical and/or nutritional acceptable derivatives thereof and any combination thereof. The above numbers are based on tocopherol equivalents, recognized in the art.

### Selenium

The present composition preferably contains selenium, because of its antioxidant activity. Preferably the present method provides the administration of a composition comprising 0.01 and 5 mg selenium per 100 ml liquid product, preferably 0.02 and 0.1 mg selenium per 100 ml liquid product. The amount of selenium administered per day is preferably more than 0.01 mg, more preferably 0.01 to 0.5 mg.

### Protein

Although the composition may further comprise proteinaceous material, it has been found that such component is not deemed necessary. In fact, it is thus possible to concentrate the actives in a low volume composition. Should a protein fraction be included, the protein fraction comprises intact proteins, peptides as may be obtained by hydrolyses of intact proteins and by syntheses, derivatives of peptides comprising more than 80 weight % amino acids. Nitrogen from nucleosides material and choline will not be calculated as being protein.

In one embodiment, it is preferred that the amount of taurine (including taurine salts) is less than 0.1 g, preferably less than 0.05 g per daily dose. Additionally or alternatively, it is preferred that the amount of taurine (including taurine salts) is less than 5 mg, more preferably less than 2.5 g per 100 g composition.

In one embodiment, the composition comprises less than 25 mg, more preferably less than 20 mg, most preferably less than 15 mg cysteine and taurine per 100 ml of the (liquid) composition. In one embodiment, the composition comprises less than 25 mg, more preferably less than 20 mg, most preferably less than 15 mg cysteine per 100 ml of the (liquid) composition. It is preferred that the protein fraction comprises more than 70 weight % of casein or caseinates, or hydrolyzates thereof, and more preferably 80 weight % or more, because caseins comprise relatively low amounts of cysteine compared to other protein sources. It is further preferred to heat the liquid composition in order to oxidize the cysteine molecules present in the protein. This impairs biological availability of any residual cysteine as present in the formula. A preferred heat treatment involves sterilization. It is preferred to maintain the temperature remains below 135° C., preferably less than 132° C. combined with a sufficient long time to have the cysteine oxidized, i.e. more than 30 seconds, preferably more than 40 seconds.

In one embodiment, it is preferred that the composition has a protein content of less than 15 en %, more preferably less than 10 en %, most preferably less than 5 en % of the total energy content of the composition. The energy percentages of the components are calculated using the calculation factors 9 kcal per g lipid, 4 kcal per g protein or g digestible carbohydrates, 2 kcal per g dietary fibers and zero kcal for the other components in the composition. In one embodiment, it is preferred that the composition comprises less than 0.5 to 10 g protein per 100 ml, more preferably less than 1 to 6 gram protein per 100 ml, most preferably 2 to 6 gram protein/100 ml.

A preferred composition according to the invention comprises, per daily dose or per 100 ml composition:
100-500 mg, preferably 200-400 mg EPA,
900-1500 mg, preferably 950-1300 mg DHA,
50-600 mg, preferably 60-200 mg phospholipids,
200-600 mg, preferably 300-500 mg choline,
400-800 mg, preferably 500-700 mg UMP (uridine monophosphate),
20-60 mg, preferably 30-50 mg vitamin E (alpha-TE),
60-100 mg, preferably 60-90 mg vitamin C,
40-80 μg, preferably 45-65 μg selenium,
1-5 μg, preferably 2-4 μg vitamin B12,
0.5-3 mg, preferably 0.5-2 mg vitamin B6, and
200-600 μg, preferably 300-500 μg folic acid.

More preferred, a composition according to the invention comprises per 100 ml composition:
100-500 mg, preferably 200-400 mg EPA,
900-1500 mg, preferably 950-1300 mg DHA,
50-600 mg, preferably 60-200 mg phospholipids,
200-600 mg, preferably 300-500 mg choline,
400-800 mg, preferably 500-700 mg UMP (uridine monophosphate),
20-60 mg, preferably 30-50 mg vitamin E (alpha-TE),
60-100 mg, preferably 60-90 mg vitamin C,
40-80 μg, preferably 45-65 μg selenium,
1-5 μg, preferably 2-4 μg vitamin B12,
0.5-3 mg, preferably 0.5-2 mg vitamin B6, and
200-600 μg, preferably 300-500 μg folic acid.

The compositions as described above can be used as a nutritional therapy, nutritional support, as a medical food, as a food for special medical purposes or as a nutritional supplement. Such product can be consumed at one, two or three servings between 75 and 200 ml per day or per unit, most preferably between 90 and 150 ml/day, most preferably about 125 mL per day in the aforementioned applications.

The subjects that can benefit from the method and composition of the invention often experience problems with eating. Their sensory capabilities and/or control of muscles can become imparted, as well as in some instances their ambition to apply proper eating habits. Swallowing and/or mastication may be problematic. Hence, the present composition is preferably provided in the form of a drink capable of being ingested through a straw.

Related therewith, the composition according to the invention preferably has a low viscosity, preferably a viscosity between 1 and 2000 mPa·s measured at a shear rate of 100 sec-1 at 20° C., more preferably a viscosity between 1 and 100 mPa·s measured at a shear rate of 100 sec-1 at 20° C. In a preferred embodiment the present composition has a viscosity of 1-80 mPa·s at a shear rate of 100 per sec at 20° C., more preferably of 1-40 mPa·s at a shear rate of 100 per sec at 20° C. These viscosity measurements may for instance be performed using plate and cone geometry.

To be optimally accepted by the subject, the present composition preferably has an osmolality of 300 to 800 mOsm/kg. However, the energy density of the product is preferably not so high that it interferes with normal eating habits. When in liquid form, the present product preferably contains between 0.2 and 3 kcal/ml, more preferably between 0.5 and 2, between 0.7 and 1.5 kcal/ml.

In one aspect, the invention pertains to a method for improving or preserving functional brain connectivity and/or functional synaptic activity and/or preserving brain network organization in a subject in need thereof, and/or slowing down, preventing or reversing impaired functional brain connectivity and/or impaired functional synaptic activity and/or impaired brain network organization of a subject in need thereof, comprising administering to said subject the composition comprising the aforementioned components (i)-(ii), and as further characterized here above.

In one aspect, the invention pertains to a method for improving or preserving functional connectivity and/or preserving brain network organization of a subject in need thereof, comprising administering to said subject the composition comprising the aforementioned components (i)-(ii), and as further characterized here above.

In one aspect, the invention pertains to a method for treating a subject in need thereof, administering, preferably at least daily, to said subject a composition comprising the aforementioned components (i)-(ii), and as further characterized here above; and monitoring said subject using an imaging technique for assessing functional connectivity, preferably one or more imaging techniques selected from the list consisting of electroencephalography (EEG), magnetoencephalography (MEG), functional magnetic resonance imaging (fMRI), fluorodeoxy glucose positron emission tomography (FDG-PET), near infra-red spectroscopy (NIRS), single-photon emission computed tomography (SPECT), arterial spin labeling (ASL), preferably EEG, fMRI and/or MEG, during treatment. In one aspect, said subject is monitored for changes in PLI. In a further aspect, the organization of functional connectivity networks can be characterized using graph theory analysis, yielding a variety of network parameters such as cluster coefficient C, characteristic path length L. In a further step, the SWI may be calculated from those parameters.

In a further aspect, the invention pertains to a composition for use in improving or preserving functional brain connectivity and/or functional synaptic activity and/or preserving brain network organization in a subject in need thereof, and/or slowing down, preventing or reversing impaired functional brain connectivity and/or impaired functional synaptic activity (in the brain) and/or impaired brain network organization of a subject in need thereof, wherein said composition comprises (i)-(ii), and as further characterized here above; and monitoring said subject with an imaging technique for assessing functional connectivity, preferably one or more imaging techniques selected from the list consisting of electroencephalography (EEG), magnetoencephalography (MEG), functional magnetic resonance imaging (fMRI), fluorodeoxy glucose positron emission tomography (FDG-PET), near infra-red spectroscopy (NIRS), single-photon emission computed tomography (SPECT), arterial spin labeling (ASL), preferably EEG and/or MEG, during treatment. In one aspect, said subject is monitored for changes in PLI. In a further aspect, the organization of functional connectivity networks can be characterized using graph theory analysis, yielding a variety of network parameters such as cluster coefficient C, characteristic path length L. In a further step, the SWI may be calculated from those parameters.

Preferably, in a method according to the invention, an increase of the PLI is indicative of a higher functional (synaptic) connectivity. In particular, an increase of the PLI in the delta band is indicative of a higher functional (synaptic) connectivity, preferably in a subject suffering from a neurodegenerative disease, more preferably in a subject suffering from AD.

In one aspect, the invention pertains to the use of an imaging technique for assessing functional connectivity and brain network organization, preferably one or more imaging techniques selected from the list consisting of electroencephalography (EEG), magnetoencephalography (MEG), functional magnetic resonance imaging (fMRI), fluorodeoxy glucose positron emission tomography (FDG-PET), near infra-red spectroscopy (NIRS), single-photon emission computed tomography (SPECT), arterial spin labeling (ASL), preferably EEG, fMRI and/or MEG for monitoring functional synaptic connectivity, functional synaptic activity and brain network organization (in particular by determining cluster coefficient C and characteristic path length L), in an intervention study, wherein said subject subjected to monitoring is provided with medication, preferably a composition comprising the aforementioned components (i)-(ii), and as further characterized herein. More details on the subject involved in the intervention study are given here above.

### EXAMPLES

### Example 1

### Packaged Composition for Comprising Per 125 ml

Energy 125 kcal; Protein 3.9 g; Carbohydrate 16.5 g; Fat 4.9 g.

Fat includes 1.5 g DHA+EPA, and 106 mg phospholipids (soy lecithin); Choline 400 mg; UMP (uridine monophosphate) 625 mg; Vitamin E 40 mg alpha-TE; Vitamin C 80 mg; Selenium 60 μg; Vitamin B12 3 μg; Vitamin B6 1 mg; Folic acid 400 μg.

Minerals and trace elements: Sodium 125 mg; Potassium 187.5 mg; Chloride 156.3 mg; Calcium 100 mg; Phosphorus 87.5 mg; Magnesium 25 mg; Iron 2 mg; Zinc 1.5 mg; Copper 225 μg; Manganese 0.41 mg; Molybdenum 12.5 μg; Chromium 8.4 μg; Iodine 16.3 μg. Vitamins: Vit. A 200 μg-RE; vit. D3 0.9 μg; vit. K 6.6 μg; Thiamin (B1) 0.19 mg; Riboflavin (B2) 0.2 mg; Niacin (B3) 2.25 mg-NE; Pantothenic acid (B5) 0.66 mg; Biotin 5 μg.

### Example 2

### Clinical Study

In the present intervention study brain network connectivity, particularly functional brain network connectivity, was investigated using electroencephalography (EEG). The study was a 24-week, randomized, controlled, double-blind study, conducted at 27 study centers. Drug-naïve patients with mild AD (MMSE scores ≧20) and diagnosis of probable AD according to the NINCDS-ADRDA criteria, were randomly assigned (1:1) to the composition including the components according to table 1, or an iso-caloric control product. The duration of intervention was 24 weeks.

TABLE 1
Nutritional composition used in clinical trials

| | Amount per | |
|---|---|---|
| component | daily dose | |
| EPA | 300 | mg |
| DHA | 1200 | mg |
| Phospholipids | 106 | mg |
| Choline | 400 | mg |
| UMP | 625 | mg |
| Vitamin E (alpha-TE) | 40 | mg |
| Vitamin C | 80 | mg |
| Selenium | 60 | μg |
| Vitamin B12 | 3 | μg |
| Vitamin B6 | 1 | mg |
| Folic acid | 400 | μg |

*125 ml, daily dose.
TE = tocopherol equivalents.

Subjects were subjected to electroencephalography (EEG) to assess ongoing oscillatory brain activity and neuronal network organization at resting state. EEG was recorded with a standard protocol for all study sites. Data were recorded on digital EEG systems from 21 electrodes at the positions of the 10-20 system: Fp2, Fp1, F8, F7, F4, F3, A2, A1, T4, T3, C4, C3, T6, T5, P4, P3, O2, O1, Fz, Cz, Pz. A common or average reference was used. Sample frequency varied between study sites (200, 256, 400, 500, 512, or 1000 Hz) and were downsampled if needed for the analyses. On-line filter settings were high pass 0.16 Hz, and low pass 70 Hz. Four 4096-sample epochs of artifact free data (containing no eye blinks, muscle artifacts, slow eye movements or ECG-artifacts) were selected from each EEG. Peak frequency was determined for the parieto-occipital electrodes as the median frequency between 4 and 13 Hz. Subsequently, peak frequency was averaged over the four epochs and over electrodes.

Functional connectivity between all electrode pairs was established with the Phase Lag Index (PLI), a measure that is relatively insensitive to volume conduction (Stam et al., 2007). Subsequently, graph graph theoretical analysis was applied to the EEG functional connectivity results. The graph represents a brain network: the electrodes are the nodes in the graph; the PLI value for a pair of nodes is the edge. From the graph, two basic network measures were computed: the mean clustering coefficient (C), and the mean shortest path length (L). C is defined by the probability that two nodes are connected when they are both connected to the same node (when they ‘share a neighbouring node’), which is a measure of local connectivity. L reflects global integration of a network and is defined by the weighted shortest path length, computed by means of Dijkstra's algorithm {VanSteen 2010}. In order to compare networks of different subjects, the networks were normalised by generating 50 random networks for each network by randomly shuffling the PLI values in each adjacency matrix. For these networks, C and L were computed. By taking the ratio (network measures of the real network divided by those of random networks), the network measures were then normalised for network size and connection strength. The resulting normalised clustering coefficient (gamma), and normalised path length (lambda) were used in further analyses.

### Results

In total, 259 patients were randomized to intervention over a 1.5 year period; 130 to the active group and 129 to the control group. The study groups were well matched with regard to all characteristics.

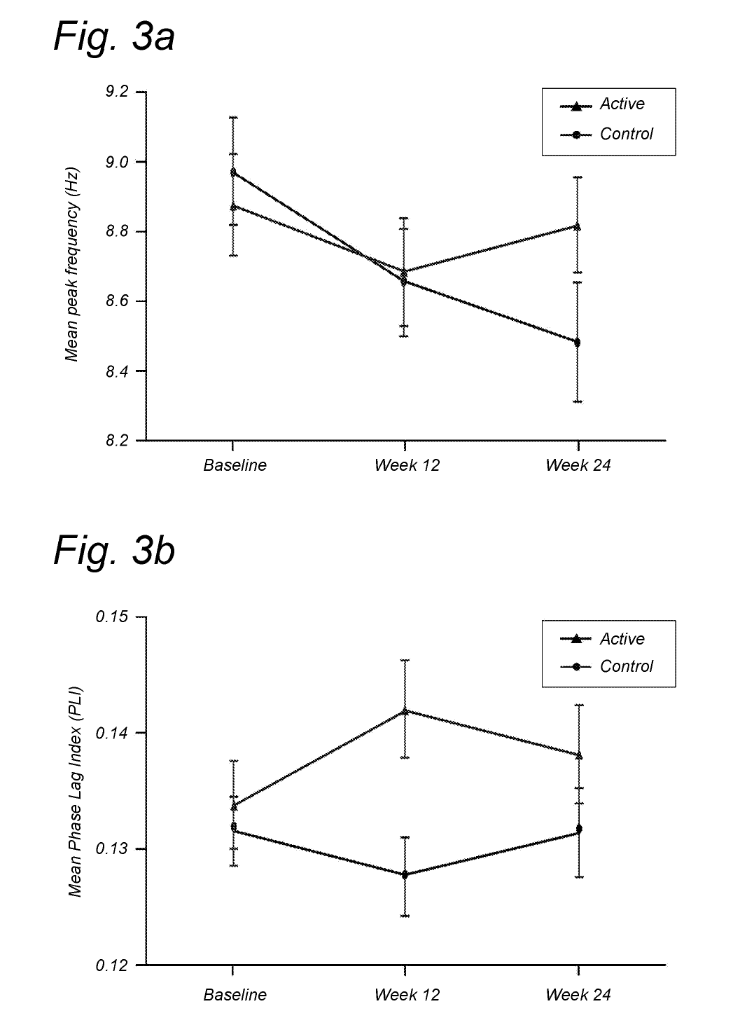

EEG data were available for a subset of 179 subjects; 86 from the active group and 93 from the control group). Functional connectivity analysis for the delta band yielded a significant intervention effect over 24-week intervention period (p=0.011). For peak frequency, the difference in trajectory over 24 weeks between the groups was significant (p=0.019). As shown in FIG. 3, peak frequency slowed in the control group during 24 weeks of intervention and remained relatively stable in the active group. The synaptic connectivity was preserved in the intervention group, while it continued to deteriorate in the control group. In summary, the study revealed significant results in terms of (1) peak frequency (p=0.019); (2) functional connectivity (PLI) in delta band (p=0.011); (3) normalized clustering coefficient (gamma) in beta band (p=0.0009); and (4) normalised path length (lambda) in beta band (p=0.053).

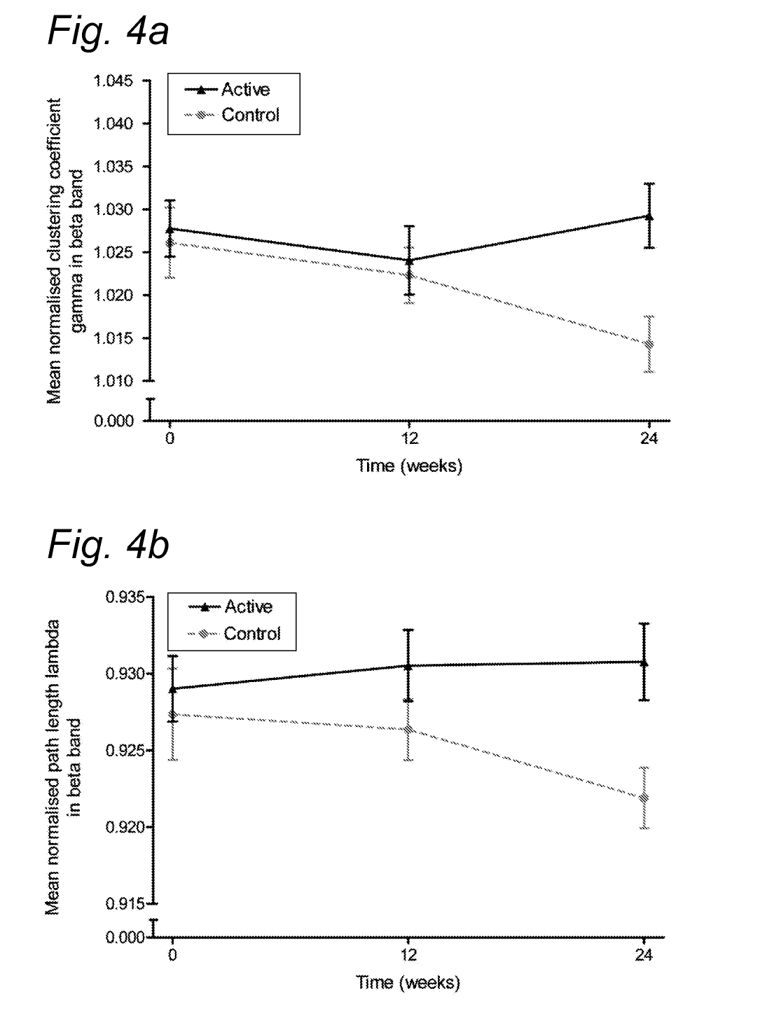

FIG. 4a depicts that mean normalized clustering coefficient C, and FIG. 4b shows the calculations for the mean normalized path length L. As illustrated in FIGS. 4a and 4b, brain networks of patients receiving control product showed a decrease in gamma (i.e. normalized clustering coefficient) and a decrease in lambda (normalized path length) in the beta band. In contrast, the patients receiving the intervention product showed stable network parameters, suggesting preserved functional brain network organisation.

The combination of decreasing local clustering and decreasing path length during the 24 weeks of the study in the control group indicates deterioration from small-world to random network organisation, as expected in progressive AD. The stable network parameters (preserved functional EEG network organization) observed in the group receiving the intervention product suggests that the product influences synaptic function and has a biological effect on the brains of patients with mild AD.

### Discussion

In conclusion, this study showed that 24-weeks of supplementation with the active composition improved functional connectivity and preserved brain network organization, and is well-tolerated in drug-naïve patients with mild AD.

### REFERENCES

Bassett D S, Bullmore E T, Human brain networks in health and disease. Curr Opin Neurol 22, 340-7 (2009).
Bozzali M et al. (2011) Regional grey matter loss and brain disconnection across Alzheimer disease evolution. Current medicinal chemistry 18:2452-2458.
Bullmore E T, Sporns O Complex brain networks: graph theoretical analysis of structural and functional systems. Nat Rev Neurosci 10, 186-98 (2009).
Cabral J, Hugues E, Sporns O, Deco G Role of local network oscillations in resting-state functional connectivity. Neuroimage 57, 130-9 (2011).
Cao X et al. (2012) Disrupted resting-state functional connectivity of the hippocampus in medication-naive patients with major depressive disorder. Journal of affective disorders.
Cubillo A, Rubia K (2010) Structural and functional brain imaging in adult attention-deficit/hyperactivity disorder. Expert Rev Neurother 10:603-620.
S, Duke T, Bullmore E T Adaptive reconfiguration of fractal small-world human brain functional networks. Proc Natl Acad Sci USA 103, 19518-23 (2006).
Gepner B, Feron F (2009) Autism: a world changing too fast for a mis-wired brain? Neurosci Biobehav Rev 33:1227-1242.
Horwitz (2003) The elusive concept of brain connectivity. Neuroimage 9, 466-470
Konrad K, Eickhoff S B (2010) Is the ADHD brain wired differently? A review on structural and functional connectivity in attention deficit hyperactivity disorder. Hum Brain Mapp 31:904-916.
Stam C J, et al. Hum Brain Mapp November, 2007; 28:1178-93.
Stam et al. Brain 132 (2009) 213-224.
Stam C J (2010) Use of magnetoencephalography (MEG) to study functional brain networks in neurodegenerative disorders. J Neurol Sci 289:128-134.
Wolf R C et al. (2008) Aberrant connectivity of lateral prefrontal networks in presymptomatic Huntington's disease. Exp Neurol 213:137-144.
Steen M Graph Theory and Complex Networks; An introduction (2010).
Watts D J, Strogatz S H, Collective dynamics of ‘small-world’ networks. Nature 393, 440-2 (1998).
Van den Heuvel (2009) J. Neurosc 29(23): 7619-24.
Lange et al (2009) Hum Brain Mapp.

1.-17. (canceled)

18. A method for improving or preserving functional brain connectivity and/or functional synaptic activity and/or brain network organization in a subject in need thereof, and/or slowing down, preventing or reversing impaired functional brain connectivity and/or impaired functional synaptic activity and/or impaired brain network organization in a subject in need thereof, the method comprising administering to the subject a composition comprising:
(a) one or more of uridine and cytidine, or salts, phosphates, acyl derivatives or esters thereof; and
(b) a lipid fraction comprising at least one of docosahexaenoic acid (22:6; DHA), eicosapentaenoic acid (20:5; EPA) and docosapentaenoic acid (22:5; DPA), or esters thereof.

19. The method according to claim 18, wherein the subject suffers from a neurological disorder.

20. The method according to claim 19, wherein the neurological disorder is a neurocognitive disorder, neurodevelopmental disorder or a depressive disorder.

21. The method according to claim 19, wherein the disorder is a neurocognitive disorder selected from the group consisting of Alzheimer's disease, Mild Cognitive impairment (MCI), Parkinson's Disease, and Huntington's Disease, or a neurodevelopmental disorder selected from the group consisting of Attention Deficit/Hyperactivity Disorder and Autism Spectrum Disorder, or a depressive disorder selected from the group consisting of depression and Chronic Depressive Disorder.

22. The method according to claim 18, wherein the subject suffers from or is at risk of a memory or cognitive disorder, memory decline or cognitive dysfunction,

23. The method according to claim 22, wherein the disorder is Age Associated Memory Impairment (AAMI), Alzheimer's Disease, multiple sclerosis, vascular dementia, frontotemporal dementia, semantic dementia or dementia with Lewy bodies.

24. The method according to claim 18, wherein the subject suffers from or is at risk of AD, dementias, MCI, memory disorders, Parkinson, obsessive compulsive disorder, Tourette's syndrome, depression, schizophrenia, Autism Spectrum Disorders (ASD), post traumatic stress syndrome (PTSD), traumatic brain injury, PKU, alcoholism, Down syndrome, epilepsy, ALS, HIV, bipolar disorder, Multiple Sclerosis, Huntington, attention-deficit/hyperactivity disorder, and autism (asperger).

25. The method according to claim 18, wherein the subject suffers from or is at risk of Alzheimer's Disease or dementia syndrome.

26. The method according to claim 25, wherein the neurodegenerative disorder is AD or dementia syndrome.

27. The method according to claim 18, wherein the composition further comprises choline, or salts or esters thereof.

28. The method according to claim 27, comprising administering 200-600 mg choline per daily dose or per 100 ml composition.

29. The method according to claim 18, wherein the composition further comprises at least one B vitamin selected from the group consisting of vitamin B6, vitamin B12 and vitamin B9.

30. The method according to claim 18, wherein the composition comprises: at least 500 mg of DHA and at least 50 mg of uridine, per 100 ml of the composition.

31. The method according to claim 18, wherein the composition comprises, per 100 ml composition:
50-1000 mg phospholipids,
0.5-3 mg vitamin B6,
50-500 μg folic acid, and
1-30 μg vitamin B12.

32. The method according to claim 18, wherein the composition comprises, per 100 ml composition:
100-500 mg EPA,
1000-1500 mg DHA,
50-600 mg phospholipids,
200-600 mg choline,
400-800 mg UMP (uridine monophosphate),
20-60 mg vitamin E (alpha-TE),
60-100 mg vitamin C,
40-80 μg selenium,
1-5 μg vitamin B12,
0.5-3 mg vitamin B6, and
200-600 μg folic acid.

33. The method according to claim 32, wherein the composition comprises, per 100 ml composition:
200-400 mg EPA,
1100-1300 mg DHA,
60-200 mg phospholipids,
300-500 mg choline,
500-700 mg UMP (uridine monophosphate),
30-50 mg vitamin E (alpha-TE),
70-90 mg vitamin C,
50-70 μg selenium,
2-4 μg vitamin B12,
0.5-2 mg vitamin B6, and
300-500 μg folic acid.

34. A method of assessing or monitoring functional brain connectivity and/or brain network organization in a subject in need thereof, comprising:
(a) administering to the subject a composition comprising:
(i) one or more of uridine and cytidine, or salts, phosphates, acyl derivatives or esters thereof; and
(ii) a lipid fraction comprising at least one of docosahexaenoic acid (22:6; DHA), eicosapentaenoic acid (20:5; EPA) and docosapentaenoic acid (22:5; DPA), or esters thereof, and
(b) subjecting the subject to an imaging technique for assessing or monitoring functional brain connectivity and/or brain network organization.

35. The method according to claim 34, wherein the imaging technique comprises electroencephalography (EEG), functional magnetic resonance imaging (fMRI) and/or magnetoencephalography (MEG).

36. A composition, comprising:
(a) one or more of uridine and cytidine, or salts, phosphates, acyl derivatives or esters thereof; and
(b) a lipid fraction comprising at least one of docosahexaenoic acid (22:6; DHA), eicosapentaenoic acid (20:5; EPA) and docosapentaenoic acid (22:5; DPA), or esters thereof.
